(19) United States
(12) Patent Application Publication
(10) Pub. No.: US 2025/0366777 A1
(43) Pub. Date: Dec. 4, 2025

(54) Data-Driven Personalized Breast CAD and Health-Tracking System

(71) Applicant: Hologic, Inc., Marlborough, MA (US)

(72) Inventors: Bibo SHI, Sunnyvale, CA (US); Ashwini KSHIRSAGAR, Cupertino, CA (US); Jin-Long CHEN, Santa Clara, CA (US); Xiangwei ZHANG, Fremont, CA (US)

(73) Assignee: Hologic, Inc., Marlborough, MA (US)

(21) Appl. No.: 18/732,415

(22) Filed: Jun. 3, 2024

(51) Int. Cl. A61B 5/00; G06T 7/00; G16H 50/20

(52) U.S. Cl. CPC A61B 5/4312; G06T 7/0014; G16H 50/20; G06T 2207/30068

(57) ABSTRACT

Systems and methods for risk-based breast cancer screening. The breast cancer screening techniques can identify and monitor women who may otherwise later be diagnosed with symptomatic and/or later-stage breast cancer. A personalized breast CAD and health-tracking system is provided that can differentiate pathological changes from normal changes in breast tomosynthesis images. A breast progression predictor can be a generative model that receives input breast images including past images captured at a past timepoint and current images captured at a current timepoint. The model uses the past images to generate predicted images for the current timepoint. Differences between the predicted images and the current images can be used to determine a likelihood of pathological change in the current images. When a pathological change is detected. The system can incorporate a broad spectrum of patient non-image information to further enhance and personalize the prediction of breast progression.

### TECHNICAL FIELD

This disclosure relates generally to personalized breast health-tracking, and more specifically, to data-driven personalized breast CAD and health-tracking systems.

### BACKGROUND

Various imaging technologies can be used to provide images of internal structures of a patient. Visualization methods can be used to screen for and diagnose cancer and other maladies in a patient. For example, early screening can detect lesions within a breast that might be cancerous so that treatment can take place at an early stage in the disease. Mammography is one type of medical imaging that generates 2-dimensional (2D) x-ray images of the breast from various angles. Tomosynthesis, also known as digital breast tomosynthesis, is a more advanced imaging technique that generates a number of images of the breast. In general, during tomosynthesis imaging, an x-ray tube moves in an arc around a breast, capturing images of the breast from different angles and also capturing images of discrete layers of the breast. The captured tomosynthesis images are reviewed by the radiologist to better visualize the breast tissue in the many discrete layers of breast. In some instances, a single synthesized image is created from tomosynthesis images to allow for quicker navigation and review of the tomosynthesis images. In other instances, slabs of images are created from the tomosynthesis images. Mammography, tomosynthesis, synthesized and slab images can be used for both screening and diagnosis of patients for cancerous lesions and other abnormalities.

### BRIEF DESCRIPTION OF THE DRAWINGS

Embodiments will be readily understood by the following detailed description in conjunction with the accompanying drawings. To facilitate this description, like reference numerals designate like structural elements. Embodiments are illustrated by way of example, and not by way of limitation, in the figures of the accompanying drawings.

FIG. 1 illustrates an example neural network, according to various examples of the disclosure;

FIG. 2 illustrates an example of a breast progression space, according to various examples of the disclosure;

FIG. 3 is a diagram illustrating an example of a breast progression predictor, according to various examples of the disclosure;

FIG. 4 is a diagram illustrating an example of a system including a breast progression predictor as well as multi-modality patient information, according to various examples of the disclosure;

FIG. 5 is a diagram illustrating an example of breast progression predictor, an input tomosynthesis image, a predicted tomosynthesis image, and a healthy breast latent manifold, according to various examples of the disclosure;

FIGS. 6A-6B show examples of a current input tomosynthesis image and a predicted tomosynthesis image, according to various examples of the disclosure;

FIG. 7A shows an example of a method for data-driven personalized CAD and health-tracking, according to various examples of the disclosure;

FIG. 7B shows an example of a method for training a data-driven personalized CAD and health-tracking system, according to various examples of the disclosure.

FIG. 8 is a block diagram of a neural network module, according to various examples of the disclosure; and

FIG. 9 is a block diagram of an example computing device, according to various examples of the disclosure.

### DETAILED DESCRIPTION

### Overview

Digital breast tomosynthesis, which generates a number of images of the breast, is widely used for routine breast cancer screening. Various artificial intelligence (AI) technologies can be used to analyze tomosynthesis images and screen the images for indications of breast cancer. While the use of AI systems can enhance the efficiency of breast cancer screening, it can also lead to an increase in false positive detections when compared to cancer screening by experienced radiologists. This is due, in part, to traditional Computer Assisted Detection and Diagnosis (CAD) systems relying solely on breast tomosynthesis and/or mammogram images. In general, traditional CAD systems use machine learning techniques for detection. In contrast, radiologists base cancer screening decisions on breast tomosynthesis (and/or mammogram) images as well as an extensive range of personalized context information, such as demographic characteristics, prior cancer history, previous screening results or treatments, and other imaging modalities. Furthermore, radiologists have access to patients' prior breast tomosynthesis and/or mammogram images and patients' medical records, which radiologists can use as direct comparative references when evaluating new screening results. These prior impressions can be instrumental in guiding radiologists to focus on specific regions of interest and helping radiologists to distinguish between pathological changes and normal aging.

In general, breast cancer risk assessment models are not image-content based. Rather, the risk assessment models rely solely on lifestyle and familial risk factors, along with mammographic density. The models are designed for longer-term risk estimation and lack the granularity to guide risk estimation for intervening time periods. However, as discussed herein, breast cancer risk assessment can be significantly more accurate when tomosynthesis-based characteristics and other personalized features are incorporated into risk estimation, thereby preventing later-stage breast cancers while also decreasing false positive screenings.

According to various implementations, systems and methods are provided herein for risk-based breast cancer screening using a conditional contextual multimodality generative model. In some examples, the risk-based breast cancer screening techniques discussed herein can identify and monitor women who, after receiving a negative or benign screening result, may benefit from supplemental and/or more intensive screening. For instance, the risk-based breast cancer screening techniques can identify and monitor women who may otherwise later be diagnosed with symptomatic breast cancer (i.e., breast cancer diagnosed between two screens) and/or later-stage breast cancer. In particular, a personalized breast CAD and health-tracking system is provided that can differentiate pathological changes from normal changes in breast tomosynthesis images. In various examples, the personalized breast CAD and health-tracking system utilizes breast screening tomosynthesis images and functions as a reference-based CAD system. The system incorporates a broad spectrum of a patient's non-image information, generates personalized predictions of breast progressions, and quantifies an age-invariant breast health index to establish a personalized breast health monitoring system.

According to various implementation, systems and methods are provided for a breast progression predictor that can evaluate breast images and differentiate normal aging changes in breasts from pathological changes. In some examples, the breast progression predictor can use a first set of breast images to predict future healthy breast images, including changes associated with normal aging. At a next timepoint, the breast progression predictor can use the predicted healthy breast images to identify pathological changes in actual images. In some examples, the breast progression predictor receives input breast images including past images captured at a past timepoint and current images captured at a current timepoint, and uses the past images to generate predicted images for the current timepoint. Differences between the generated predicted images and the current images can be identified and used to determine a likelihood of pathological change in the current images. Additionally, when a pathological change is detected, the breast progression predictor can determine a probable rate of progress of the pathological change, which, for example, can indicate whether the detected pathology is fast-acting or slow-acting.

In various implementations, the breast progression predictor can be a neural network. In some examples, the breast progression predictor is a generative model, which generates the predicted future images. In some examples, the breast progression predictor uses an age diffusion module to predict the future images. The breast progression predictor can be trained using sets of breast images, with each set including first images captured at a first timepoint and second images captured at a second timepoint. Using many sets of breast images of healthy breasts encoded into latent vectors, a healthy breast latent manifold is generated by encoding the images at each time point into a latent vector, and generating a smooth curve between the latent vectors at each time point in a set of images. The breast progression predictor can use the healthy breast latent manifold to generate the predicted images of healthy breasts and identify images with pathological change.

While the description refers generally to breast tomosynthesis or tomosynthesis images throughout, it is understood that in various implementations the systems and methods described herein could apply to other images or combinations of other images.

For purposes of explanation, specific numbers, materials and configurations are set forth in order to provide a thorough understanding of the illustrative implementations. However, it will be apparent to one skilled in the art that the present disclosure may be practiced without the specific details or/and that the present disclosure may be practiced with only some of the described aspects. In other instances, well known features are omitted or simplified in order not to obscure the illustrative implementations.

Further, references are made to the accompanying drawings that form a part hereof, and in which is shown, by way of illustration, embodiments that may be practiced. It is to be understood that other embodiments may be utilized, and structural or logical changes may be made without departing from the scope of the present disclosure. Therefore, the following detailed description is not to be taken in a limiting sense.

Various operations may be described as multiple discrete actions or operations in turn, in a manner that is most helpful in understanding the claimed subject matter. However, the order of description should not be construed as to imply that these operations are necessarily order dependent. In particular, these operations may not be performed in the order of presentation. Operations described may be performed in a different order from the described embodiment. Various additional operations may be performed or described operations may be omitted in additional embodiments.

For the purposes of the present disclosure, the phrase “A or B” or the phrase “A and/or B” means (A), (B), or (A and B). For the purposes of the present disclosure, the phrase “A, B, or C” or the phrase “A, B, and/or C” means (A), (B), (C), (A and B), (A and C), (B and C), or (A, B, and C). The term “between,” when used with reference to measurement ranges, is inclusive of the ends of the measurement ranges.

The description uses the phrases “in an embodiment” or “in embodiments,” which may each refer to one or more of the same or different embodiments. The terms “comprising,” “including,” “having,” and the like, as used with respect to embodiments of the present disclosure, are synonymous. The disclosure may use perspective-based descriptions such as “above,” “below,” “top,” “bottom,” and “side” to explain various features of the drawings, but these terms are simply for ease of discussion, and do not imply a desired or required orientation. The accompanying drawings are not necessarily drawn to scale. Unless otherwise specified, the use of the ordinal adjectives “first,” “second,” and “third,” etc., to describe a common object, merely indicates that different instances of like objects are being referred to and are not intended to imply that the objects so described must be in a given sequence, either temporally, spatially, in ranking or in any other manner.

In the following detailed description, various aspects of the illustrative implementations will be described using terms commonly employed by those skilled in the art to convey the substance of their work to others skilled in the art.

The terms “substantially,” “close,” “approximately,” “near,” and “about,” generally refer to being within +/−20% of a target value as described herein or as known in the art. Similarly, terms indicating orientation of various elements, e.g., “coplanar,” “perpendicular,” “orthogonal,” “parallel,” or any other angle between the elements, generally refer to being within +/−5-20% of a target value as described herein or as known in the art.

In addition, the terms “comprise,” “comprising,” “include,” “including,” “have,” “having” or any other variation thereof, are intended to cover a non-exclusive inclusion. For example, a method, process, device, neural network, or imaging system that comprises a list of elements is not necessarily limited to only those elements but may include other elements not expressly listed or inherent to such method, process, device, neural network, or imaging system. Also, the term “or” refers to an inclusive “or” and not to an exclusive “or.”

The systems, methods and devices of this disclosure each have several innovative aspects, no single one of which is solely responsible for all desirable attributes disclosed herein. Details of one or more implementations of the subject matter described in this specification are set forth in the description below and the accompanying drawings.

### Example Personalized Breast CAD and Health-Tracking System

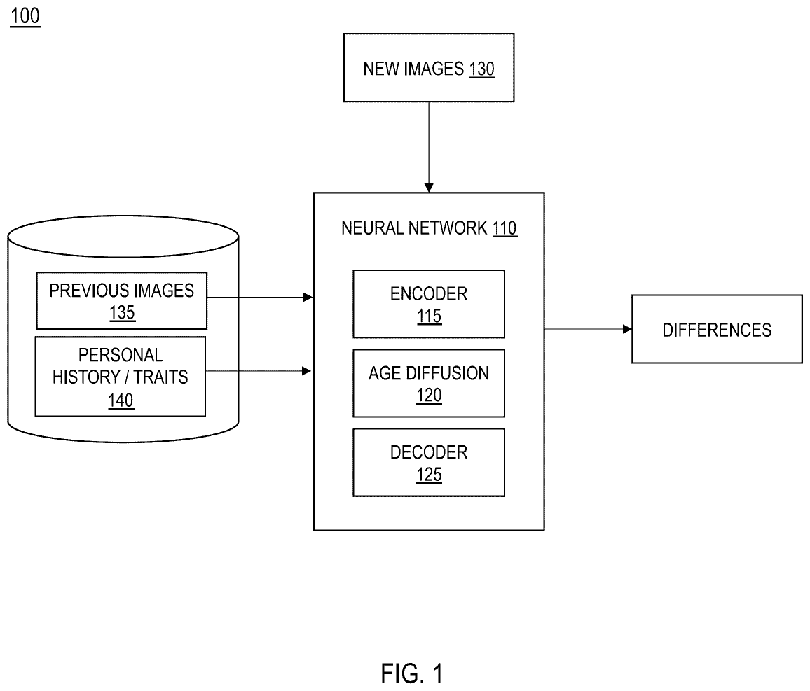

FIG. 1 illustrates an example of a personalized breast CAD and health-tracking system 100, according to various examples of the disclosure. The system 100 includes a neural network 110 that acts as a breast progression predictor. The neural network 110 predicts how healthy breasts will appear in future screening images. In particular, in some examples, the neural network 110 can predict normal breast age-related changes in a first set of breast tomosynthesis images, and, based on the predictions, the neural network 110 can differentiate pathological change from normal aging in a next set of breast tomosynthesis images. In various examples, for a particular patient, the neural network 110 receives input previous breast tomosynthesis images 135 from one or more previous imaging sessions of the patient, and, based on the input images 135 from the one or more previous imaging sessions, the neural network 110 generates a personalized prediction of breast progression for the patient. The personalized prediction of breast progression can include predicted tomosynthesis images at future points in time. In various implementations, the neural network 110 constructs a healthy breast progression latent curve for the patient, including previous tomosynthesis images 135 from the one or more previous imaging sessions and the predicted future tomosynthesis image data.

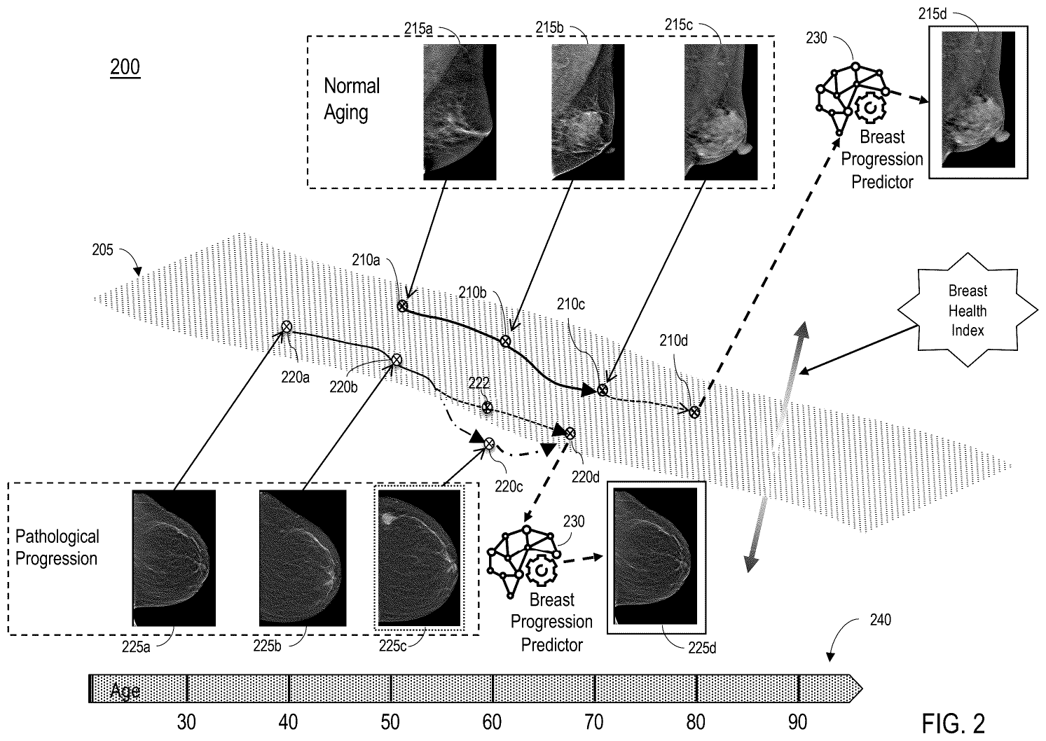

The neural network 110 includes an encoder 115, an age diffusion module 120, and a decoder 125. The encoder 115 receives the input breast tomosynthesis images 135 from one or more previous imaging sessions of the patient and encodes each image into a latent vector R″. In various examples, when the input images 135 include images at more than one timepoint, the encoder 115 encodes the input images 135 as a smooth curve in the healthy breast progression latent space, and the latent vectors become part of a healthy breast latent manifold as described with respect to FIG. 2. The smooth curve generated by the encoder 115 can have a map function ƒ: R³ à Rⁿ. The age diffusion module 120 uses the encoded images and the smooth curve to generate predicted future tomosynthesis images by interpolation. The age diffusion module 120 can be customized to perform a selected number of age diffusion cycles, where the number of age diffusion cycles determines the time gap between the most recent input image and the generated predicted future image. In particular, a greater number of age diffusion cycles is used to generate a predicted future image that has a gap of a greater number of years from the most recent input image. In some examples, the decoder 125 decodes a latent point along the smooth curve, where the latent point represents a predicted future tomosynthesis image. In particular, the decoder 125 decodes the latent point into a predicted future tomosynthesis image.

In various examples, the latent point decoded by the decoder 125 corresponds to the point along the smooth curve at which the new tomosynthesis images 130 are captured. The neural network 110 compares the predicted future tomosynthesis images generated by the decoder 125 with the new input images 130. Differences between the predicted future tomosynthesis images and the new input images 130 can indicate pathological change. In some examples, the new input images 130 can be encoded by the encoder 115 to generate an encoded latent point for the new input images 130. If the encoded latent point is not on the healthy breast latent manifold, the neural network 110 can determine that the new input images 130 indicate a pathological change that is not due to normal aging. In some examples, if the encoded latent point for the new input images 130 is on the healthy breast latent manifold but veering off towards an edge of the manifold, the neural network 110 can identify an early risk for potential future pathological change. Similarly, if the encoded latent point for the new input images 130 is on the healthy breast latent manifold but veering away from a predicted patient healthy breast latent curve, the neural network 110 can identify an early risk for potential future pathological change. In some examples, when an early risk for potential future pathological change is identified, the patient can be instructed to return for follow-up at an earlier date and potential future pathological abnormalities can be caught early, preventing late-stage disease.

Additionally, in various examples, the neural network 110 can receive personal history and/or personal trait data 140 for the patient. In some examples, the personal history and/or personal trait data 140 for the patient can include some or all of a patient's electronic health record. The personal history and/or personal trait data 140 for the patient can be used by the neural network 110 to generate personalized predictions of breast progression. In some examples, the personal history and/or personal trait data 140 for the patient can be used to quantify an age-invariant breast health index that is used in establishing the patient's healthy breast latent manifold and/or the smooth curve in the health breast progression latent space for the patient.

According to various implementations, the neural network 110 is a generative model, and, in various examples, the neural network 110 can be a generative diffusion model. The neural network 110 constructs a breast progression latent space which represents the normal aging curves of breasts from multiple different patients. In some examples, one or more additional neural network models can be used in the breast progression predictor, for instance, for encoding the images into latent vectors, decoding the predicted latent vectors into images, identifying differences between predicted images and captured images, for determining risk, etc.

According to various implementations, the neural network 110 can be used on other types of images and/or on images of other body parts, organs, whole body imaging, etc. For instance, the neural network 110 can be used on images of lungs, heart images, liver images, and/or on images of other body part and organs. In some examples, the neural network 110 can be used to evaluate DEXA scan images, bone density scan images, CT scan images, MRI images, PET scan images, ultrasound images, radiographic images, or other types of images. In some examples, the neural network 110 can be used to evaluate any type of mammography images, such as 2D images, synthesized images, stacks of images, slabs images such as those generated by the 3DQuorum® technology manufactured by Hologic, Inc., and so on.

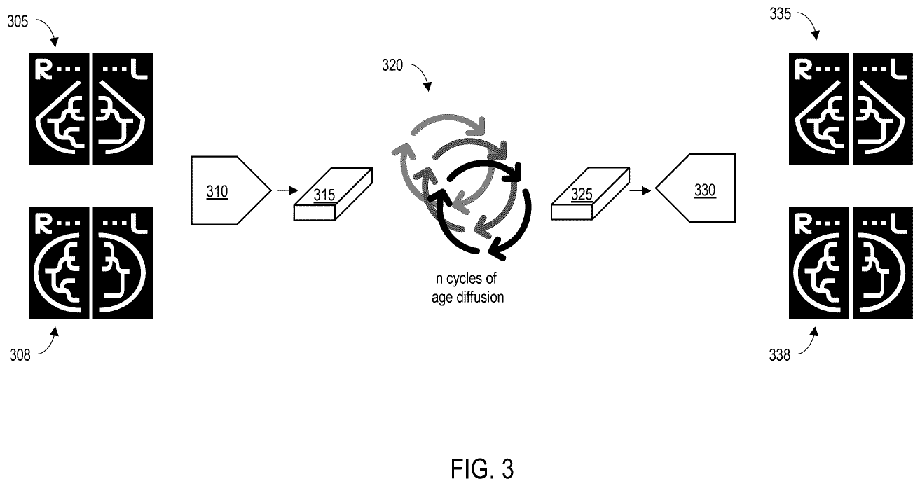

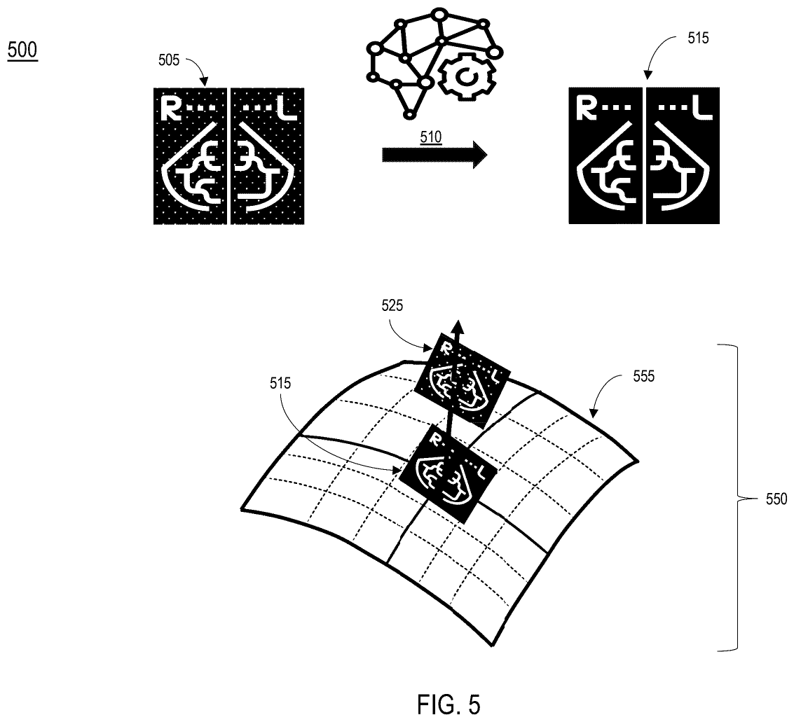

FIG. 2 illustrates an example 200 of a breast progression space, according to various examples of the disclosure. In particular, the breast progression space is mathematically a topological ambient space (Rⁿ), on which the progression for each female's breasts can be encoded as a smooth curve using a map function (f: Rⁿà Rⁿ) to generate a healthy breast latent manifold 205. The healthy breast latent manifold 205 is generated from the normal aging curves of breasts from many different female cases. In particular, tomosynthesis images collected over time can be encoded and mapped into a sequence of high dimensional latent points used to generate the healthy breast latent manifold 205. A sequence of high dimensional latent points, each point representing tomosynthesis images at a particular point in time for a particular patient, can be connected via a smooth curve on the healthy breast latent manifold 205. Thus, in various examples, any point on the healthy breast latent manifold 205 can be decoded and mapped back to a breast tomosynthesis image for a selected patient at a selected age at which the corresponding breast is healthy. The healthy breast latent manifold 205 represents healthy breasts at various ages, with latent points mapped by age from one end of the manifold (e.g., age 20) to the other end of the manifold (e.g., age 100). In the example 200 of FIG. 2, age progresses from left to right, as indicated by the age progression arrow 240. Thus, latent points representing the youngest breasts are shown on the left hand side of the healthy breast latent manifold 205, and the latent points representing the oldest breasts are shown at the right hand side of the healthy breast latent manifold 205. In general, the healthy breast latent manifold 205 quantifies an age-invariant breast health index and can be used to establish a personalized breast health monitoring system. In some examples, a patient breast health index can be quantified as described below with respect to FIG. 5. Training of the breast progression predictor and generation of the healthy breast latent manifold 205 is discussed in greater detail below, for example with respect to FIGS. 3, 4, and 7B.

When current tomosynthesis images are collected for a patient, the patient's current and previous tomosynthesis images can be encoded. Referring to FIG. 2, a first patient's previous 215a, 215b and current 215c encoded tomosynthesis images can be mapped onto a sequence of high dimensional latent points 210a, 210b, 210c on the healthy breast latent manifold 205. The points 210a, 210b, 210c can be connected via a smooth curve on the healthy breast latent manifold 205. Similarly, a second patient's previous 225a, 225b and current 225c encoded tomosynthesis images can be mapped onto a sequence of high dimensional latent points 220a, 220b, 220c. For the second patient, the high dimensional latent point 220c corresponding to the current tomosynthesis image 225c is not lying on the healthy breast latent manifold 205, indicating pathological abnormalities in the breast. In particular, pathological abnormalities in the breast can be directly reflected in the tomosynthesis image 225c, and the abnormalities are reflected in the encoded latent point 220c. In various examples, a personalized breast CAD and health-tracking system, such as the system 100, can use a healthy breast latent manifold 205 and determine that the current tomosynthesis image 225c includes pathological abnormalities. In some examples, the personalized breast CAD and health-tracking system can flag the current tomosynthesis image 225c as abnormal and potentially pathological. In some examples, as described in greater detail below, instead of directly encoding the images 225c to a latent point 220c, a breast progression predictor identifies the predicted latent point 222 where the latent point 220c should lie on the manifold if the breast were healthy, generates a predicted image corresponding to the latent point 222, and then identifies differences between the predicted image and the image 225c.

According to various implementations, a breast progression predictor 230 can use the input images and the healthy breast latent manifold 205 to determine how future healthy breast tomosynthesis images for a patient will appear at a certain age. i.e., during next year's screening. As shown in FIG. 2, for the first patient, the breast progression predictor 230 uses the healthy breast latent manifold 205 and the high dimensional latent points 210a, 210b, 210c to identify the smooth curve connecting the high dimensional latent points 210a, 210b, 210c, and identify the predicted future high dimensional latent point 210d. The breast progression predictor 230 decodes the predicted latent point 210d to generate the corresponding predicted future tomosynthesis image 215d for the first patient.

For the second patient, the breast progression predictor 230 uses the healthy breast latent manifold 205 and the high dimensional latent points 220a, 220b, 220c. The breast progression predictor 230 can extrapolate a normal aging curve using the latent points 220a, 220b to identify the smooth curve connecting the latent points 220a, 220b and generate the predicted healthy breast latent point 222. The high dimensional latent point 220c, which falls off the healthy breast latent manifold, is used to identify a pathological progression curve.

According to various examples, with effective interventions (i.e., hormone treatments, surgeries, use of implantable markers, etc.), the second patient can be fully recovered and her breasts will be healthy in the future. The breast progression predictor 230 uses the pathological progression curve and the extrapolated normal aging curve to identify the future high dimensional latent point 220d for the second patient. The breast progression predictor 230 decodes the predicted latent point 220d to generate the corresponding predicted future tomosynthesis image 225d for the second patient.

According to some implementations, a breast progression predictor 230 can use the input images and the healthy breast latent manifold 205 to determine that future breast tomosynthesis images for a patient are predicted to fall off the healthy breast latent manifold 205. That is, Thus, the breast progression predictor 230 can be used to identify patients who should receive additional screenings to catch early pathological abnormalities and prevent late stage disease.

FIG. 3 is a diagram illustrating an example of a breast progression predictor 300, according to various examples of the disclosure. In various examples, the breast progression predictor 300 is a generative model, which encodes received tomosynthesis images into latent vectors, predicts latent vectors corresponding to future tomosynthesis images, and decodes the predicted latent vectors back into realistic tomosynthesis images. The breast progression predictor 300 receives input tomosynthesis images 305. In various examples, the input tomosynthesis images 305 can include images from one or more selected time points. In some examples, the tomosynthesis images 305 are previously recorded tomosynthesis images of a patient's breasts, for example tomosynthesis images from one or more years ago. The tomosynthesis images 305 can include multiple tomosynthesis images of a patient's breasts at a first selected time point and multiple tomosynthesis images of a patient's breasts at a second selected time point.

The tomosynthesis images 305, 308 are received at the breast progression predictor 300 and input to an image encoder 310. The image encoder 310 encodes the tomosynthesis images 305, 308 into one or more high dimensional latent vectors 315 for each breast. In some examples, the image encoder 310 encodes multiple tomosynthesis images 305, 308 from a selected imaging session (a selected time point) into a single latent vector 315 for each breast. In some examples, the tomosynthesis images 305 include bilateral mediolateral oblique (MLO) views and the images 308 include bilateral craniocaudal (CC) views. In some examples, the input tomosynthesis images 305, 308 include additional views, such as mediolateral views, lateromedial views, lateromedial oblique views, or other types of supplementary views. In some examples, the tomosynthesis images 305, 308 include images from multiple imaging sessions (i.e., multiple selected time points), and the image encoder 310 encodes the respective tomosynthesis images 305, 308 from each respective imaging session (i.e., each respective time point) and for each breast into a respective latent vector 315, generating a latent vector 315 for each imaging session (i.e., for each selected time point) for each breast. The image encoder 310 outputs the one or more high dimensional latent vectors 315 to an age diffusion module 320.

The age diffusion module 320 can be a generative model, and in some examples, the age diffusion module 320 can be a latent diffusion model. The age diffusion module 320 predicts future tomosynthesis images corresponding to the input tomosynthesis images 305, 308. In particular, the age diffusion module 320 predicts future latent vectors corresponding to the one or more latent vectors 315. The age diffusion module 320 can use a healthy breast latent manifold, such as the healthy breast latent manifold 205, to predict the future latent vectors. Note that the age diffusion module 320, and the effects of a given diffusion cycle, are age specific. In particular, the healthy breast latent manifold is constructed from latent vectors at selected timepoints, where each timepoint corresponds to a patient's age. Using the healthy breast latent manifold, the age diffusion module 320 maps the one or more latent vectors 315 to the latent manifold based on the patient age corresponding to timepoint at which the tomosynthesis images 305 were captured. The number of age diffusion cycles performed by the age diffusion module controls the time gap between the input tomosynthesis images 305 and the predicted future tomosynthesis images. In some examples, each age diffusion cycle corresponds to one year of aging. Thus, for n=1 (where n is the number of age diffusion cycles), the generated predicted future tomosynthesis image corresponds to how the breasts imaged in the input tomosynthesis images 305 will appear in images one year in the future. Similarly, for n=2, the generated predicted future tomosynthesis image corresponds to how the breasts imaged in the input tomosynthesis images 305 will appear two years in the future.

The age diffusion module 320 outputs a predicted future latent vector 325 corresponding to the selected number of years of aging. A decoder 330 decodes the predicted future latent vector 325 and generates a corresponding predicted future tomosynthesis image 335. In some examples, the decoder 330 uses learned weights to generate the predicted future tomosynthesis image 335 from the predicted future latent vector 325.

According to various implementations, the breast progression predictor 300 is trained using paired tomosynthesis images at different time points from multiple patients. A tomosynthesis image pair includes a first set of tomosynthesis images of a selected patient at a first time point and a second set of tomosynthesis images of the selected patient at a second time point. In some examples, the first and second time points are one year apart, and in some examples, the first and second time points are more than one year apart. In some examples, the tomosynthesis image pair used for training includes images of healthy breasts at both the first and second time points. Images of healthy breasts can be used to train the breast progression predictor 300 to recognize healthy breasts and to predict healthy age-related changes in breast images.

During training, the breast progression predictor 300 begins with tomosynthesis image pairs having a one-year time gap between the first and second sets of tomosynthesis images. For each tomosynthesis image pair, the breast progression predictor 300 ingests the first set of tomosynthesis images, encodes the first set of tomosynthesis images into a first latent vector, performs one cycle of age diffusion on the first latent vector to generate a predicted latent vector, and decodes the predicted latent vector back into the image domain, generating predicted future tomosynthesis images. The predicted future tomosynthesis images can be compared to the second set of tomosynthesis images of the image pair, where the second set of tomosynthesis images are considered ground truth images. Similarly, the predicted latent vectors corresponding to future tomosynthesis images can be compared to the second latent vectors for the second set of tomosynthesis images of the image pair, where the second set of tomosynthesis images are considered ground truth images During training, the breast progression predictor 300 can use the comparison as feedback to adjust the age diffusion module predictions. Note that the comparison can be a comparison of the tomosynthesis images and/or the comparison of the latent vectors. In some examples, the breast progression predictor 300 generates new predicted future tomosynthesis images based on the feedback, and the new predicted future tomosynthesis images are compared to the ground truth images. In various examples, during training, the breast progression predictor 300 can go through multiple cycles of training with each pair (and/or sequence) of images. In some examples, the latent diffusion model learns a diffusion process using a Gaussian process including multiple cycles of transformations to generate a predicted image that is closer to a ground truth image.

In various implementations, the breast progression predictor 300 is also trained with tomosynthesis image pairs having more than a one-year time gap. In some examples, the breast progression predictor 300 is trained with image sequences including multiple sets of images at various time points. In various implementations, an image sequence can include sets of images at many time points. For instance, an image sequence can include a first set of tomosynthesis images of a selected patient at a first time point, a second set of tomosynthesis images of the selected patient at a second time point, a third set of tomosynthesis images of the selected patient at a third time point. In some examples, the second time point is one year after the first time point, and third time point is one year after the second time point. In other examples, there is a larger gap between two or more of the time points.

According to various implementations, upon convergence, the predicted future images generated by the age diffusion module 320 have minimal differences from the ground truth images. In some examples, age diffusion module 320 can be fine-tuned for multiple year time gaps using the chain rule:

$${t}_{x + 1} = f ( {t}_{x 0} ) , {t}_{x + 2} = f ( {t}_{x + 1} ) = f ( f ( {t}_{x 0} ) )$$

In some examples, the breast progression predictor 300 can be trained to recognize abnormal breast images and to predict abnormal breast changes. In particular, the breast progression predictor 300 is trained using images of healthy breasts and generates the healthy breast latent manifold based on the healthy breast images, and in some examples, the breast progression predictor 300 can be trained to identify input images that are different from predicted images, and/or to identify input images that are not on the healthy breast latent manifold. In some examples, various pathological changes can include indications of progression speed and risk level, such that the breast progression predictor 300 can be trained to assign a risk level and an expected progression speed to detected pathological changes.

In various implementations, at the age diffusion module 320, during training, pairs of normal images (images of healthy breasts) are distinguished from pairs of images with pathological changes (images of abnormal breasts). This allows the age diffusion module 320 to generate a healthy breast latent manifold having a smooth hyperplane. The smooth hyperplane includes the encoded latent vectors of selected image pairs and/or image sequences connected to form the shortest path along the smooth hyperplane, where selected image pairs and/or sequences represent images of the same breasts taken at different time points. In various examples, any point on the healthy breast latent manifold can be decoded and mapped back to one or more breast tomosynthesis images of healthy breasts for a selected patient at a selected age. In some examples, the point can be a point in the past and the breast tomosynthesis images of the healthy breasts can be recorded as encoded input images, and in some examples, the point can be a point in the future and the breast tomosynthesis images of the healthy breasts can be predicted images.

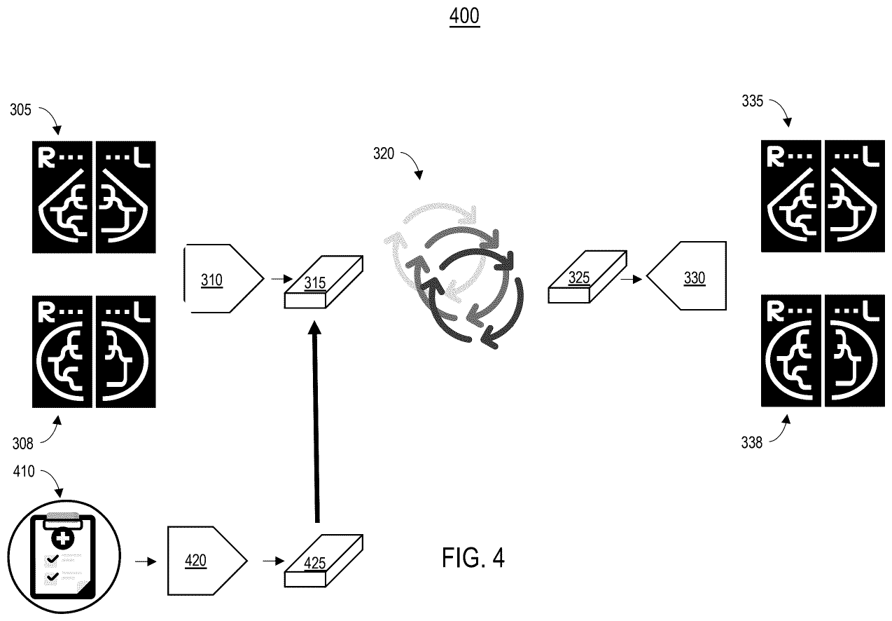

FIG. 4 is a diagram illustrating an example of a system 400 including the breast progression predictor 300 as well as multi-modality patient information 410, according to various examples of the disclosure. In various examples, the breast progression predictor 300 is infused with the patient information 410, which is used by the model to generate more accurate and personalized predictions for the respective patient. The breast progression predictor 300 is generally provided with the patient age along with the input tomosynthesis images 305. The patient information 410 can include basic patient information such as patient race, ethnicity, and relevant family history. The patient information 410 can be derived from the patient's electronic health record, and can include prior radiology screening reports, diagnostic reports, treatment reports, and other related documents. The patient information 410 can be used by the breast progression predictor 300 to adjust the predicted future images. For instance, if the patient is taking hormonal therapy, the treatment can cause breast density to increase and thus the predicted normal (healthy) future image prediction is changed accordingly to reflect breasts with increased density.

In various examples, the patient information 410 is encoded at the encoder 420. The encoder 420 can embed the patient information 410 into a latent vector 425. The patient information 410 can be text-based information and the encoder 420 can be a large language model (LLM). Thus, the encoder 420 can interpret the text-based patient information 410 and embed the information into the latent vector 425. The breast progression predictor 300 can use the latent vector 425 in combination with the one or more high dimensional latent vectors 315 to generate the predicted future latent vector 325. In some examples, conditional image generation (CIG) is used to combine the latent vector 425 with the one or more high dimensional latent vectors 315. The breast progression predictor 300 can be trained to incorporate the non-image related information about each patient embedded in the latent vector 425 in generating the predicted future latent vector 325. In some examples, a conditional control is added into the training of the breast progression predictor 300 to train the breast progression predictor 300 in conditional image generation. In some examples, the system 400 offers more personalized predictions and thus more accurate predictions.

FIG. 5 is a diagram illustrating an example 500 of breast progression predictor 510, a previous tomosynthesis image 505, a predicted tomosynthesis image 515, an input current tomosynthesis image 525, and a healthy breast latent manifold 555, according to various examples of the disclosure. In various examples, the predicted tomosynthesis image 515 is the predicted image for the time at which the input current image 525 is captured. The breast progression predictor 510 receives the previous tomosynthesis image 505. In some examples, the breast progression predictor receives additional previous tomosynthesis images from other previous years. In some examples, the breast progression predictor 510 also receives other patient information as described with respect to FIG. 4. The breast progression predictor 510 uses the previous tomosynthesis image 505 to generate a predicted tomosynthesis image 515 on the healthy breast latent manifold 555. The input current tomosynthesis image 525 is compared to the predicted tomosynthesis image 515, and unexpected differences between the predicted image 515 and the current image 525 can be highlighted. The presence of differences between the predicted image 515 and the current image 525 can be presented visually using a schematic 550 to illustrate a distance between the current image 525 and the healthy breast latent manifold 555.

In some examples, the distance between the current image 525 and the healthy breast latent manifold 555 at the age corresponding to the timepoint the current image 525 was captured can be used in determining a risk profile for the patient, such as a risk percentage or rating (e.g., high risk, medium risk, low risk). In particular, as discussed for example with respect to FIGS. 2-4, the current image 525 can be encoded as a latent vector, and the breast risk can be defined as the tangent distance between the encoded latent vector and the healthy breast latent manifold 555. In some examples, when the encoded latent vector lies on the healthy breast latent manifold, the risk is zero, indicating a healthy breast. When the encoded latent vector lies far from the manifold, the risk is very high, indicating pathological progression for the breast. In some examples, a breast health index can be quantified as one over the distance between the encoded latent vector and the healthy breast latent manifold 555. In some examples, when there is a risk of pathological abnormalities, the risk can include a predicted speed of pathological progression. The breast health and/or breast risk system can be based on a healthy breast latent manifold that is trained using multimodal patient information from patients at many different ages, and the system is able to predict healthy breast images for many different ages and detect pathological breast changes at many different ages.

### Example User Interface for Personalized Breast CAD and Health-Tracking

The personalized breast CAD and health-tracking system can present breast image data to a user, such as to a radiologist. In some examples, when potential pathological changes are detected in input images, the areas of potential concern can be highlighted in displayed images. In some examples, when a breast progression predictor generates a predicted image including normal age-related changes, and the breast progression predictor determines that the current (actual) image includes pathological changes that are different from the predicted image, both the predicted image and the current (actual) image can be displayed to a user. In some examples, an animated image is displayed to the user, in which the image progresses from the predicted image to the current (actual) image, and the animation helps illustrate the differences between the predicted image and the current image.

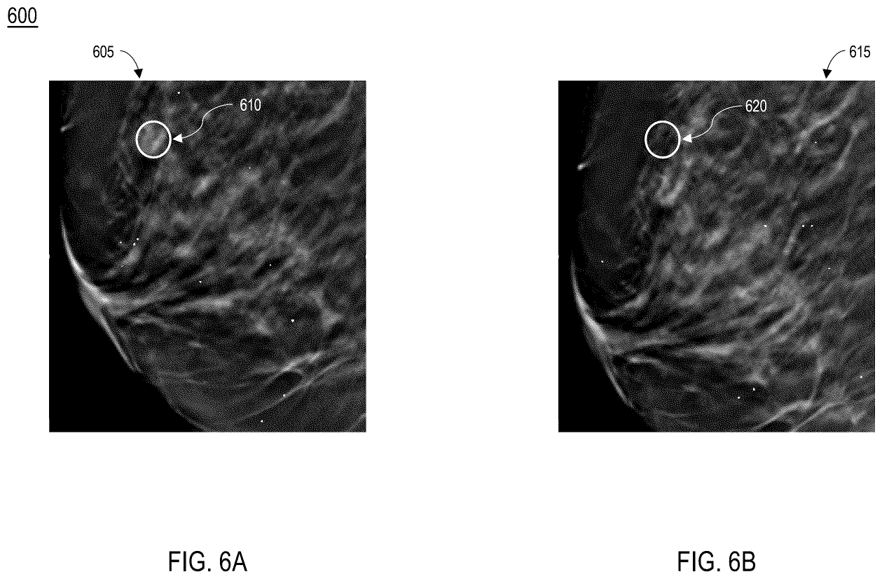

FIG. 6A shows an example of a current input tomosynthesis image 605, and FIG. 6B shows an example of a predicted tomosynthesis image 615 (which may have been generated based on previous images), according to various examples of the disclosure. As shown in FIGS. 6A and 6B, differences between the input tomosynthesis image 605 and the predicted tomosynthesis image 615 can be highlighted for a user. In particular, in FIG. 6A, a hotspot 610 is highlighted in the current input tomosynthesis image 605, where the hotspot 610 is an area of the input image 605 in which the input image is different from a corresponding area 620 of the predicted image 615. In FIG. 6B, the corresponding area 620 is highlighted. A user can use hotspot indicators such as the circles identifying the hotspot 610 and the corresponding area 620 to identify differences between the input image 605 and the predicted image 615. In some examples, a radiologist or other user evaluating the current input image 605 can pay particular attention to the identified hotspot 610 and analyze the hotspot 610 for potential abnormalities and/or pathologies.

In some examples, the hotspot 610 can be analyzed by the breast progression predictor, and the breast progression predictor can quantify the differences between the predicted tomosynthesis image 615 at the corresponding area 620 and the current input tomosynthesis image 605 at the hotspot 610. In some examples, the breast progression predictor can estimate breast health and/or breast risk using a latent vector corresponding to the current tomosynthesis image 605 and a healthy breast latent manifold. In various examples, the breast risk (e.g., the risk level of any current pathological changes, as well as the risk a breast will develop a pathological change in the future) can also be displayed to the user. The breast risk can be displayed as a distance of the latent vector from the manifold (i.e., as shown in FIG. 2 and/or in the schematic 550 of FIG. 5). In some examples, the risk can be an overall risk for each breast. In some examples, the breast risk can include a separate risk for various regions of each breast. In some examples, the breast risk can include an estimated speed of progression. For instance, pathological changes detected can be at low risk of progression and/or predicted progression of pathological changes can be slow. Conversely, pathological changes detected can be at high risk of progression and/or predicted progression of pathological changes can be fast. This data can be presented to the user as a risk profile along with the image data.

### Example Methods of Personalized Breast CAD and Health-Tracking

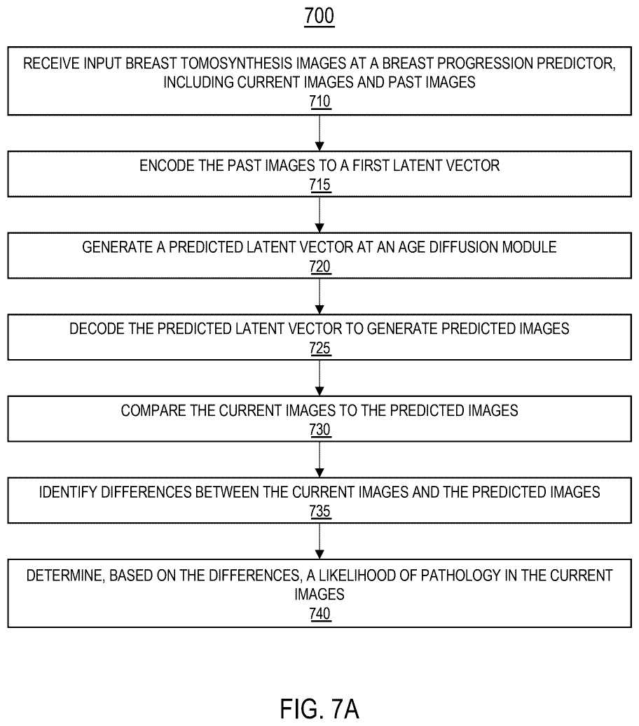

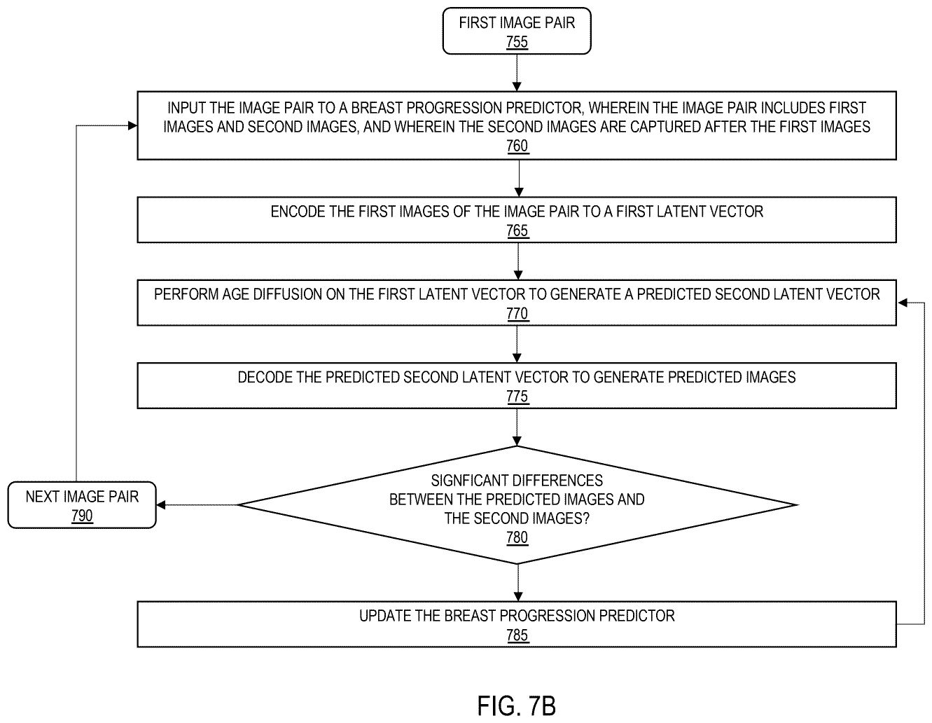

FIG. 7A shows an example of a method 700 for data-driven personalized CAD and health-tracking, according to various examples of the disclosure. In particular, FIG. 7A shows a method for personalized CAD and health-tracking of breast health using a breast progression predictor and breast tomosynthesis images as described with respect to FIGS. 1-6B. The method 700 may be performed by the neural networks described herein. Although the method 700 is described with reference to the flowchart illustrated in FIG. 7A, many other methods for data-driven personalized CAD and health-tracking may alternatively be used. For example, the order of execution of the steps in FIG. 7A may be changed. As another example, some of the steps may be changed, eliminated, or combined.

At step 710, breast tomosynthesis images for a selected patient are received at a breast progression predictor. The breast tomosynthesis images include current images and past images. In particular, the breast tomosynthesis images include past images of the select patient's breasts captured at a first time point and current images of the select patient's breast captured at a second time point. In some examples, there is a one year time interval between the first time point and the second time point. In other examples, the time interval can be less than one year or greater than one year.

Next, predicted images are generated based on the past images. At step 715, the past images are encoded in a first latent vector. In some examples, the breast progression predictor is a generative model that includes an encoder which encodes the past images to a first latent vector.

At step 720, a predicted latent vector is generated at an age diffusion model. In some examples, the breast progression predictor processes the first latent vector at an age diffusion module to generate a predicted latent vector for a selected time point corresponding to the second time point. In some examples, the age diffusion module uses a healthy breast latent manifold to generate the predicted latent vector. Thus, the predicted latent vector represents a predicted image of a healthy breast including normal age-related changes. The age diffusion module can process the first latent vector using a selected number of age diffusion cycles, where the selected number of age diffusion cycles corresponds to the time interval between the first time point and the second time point. For instance, one age diffusion cycle can correspond to one year of aging, such that a one year time interval corresponds with one age diffusion cycle at the age diffusion module. In other examples, each age diffusion cycle can correspond to a different time interval, such as a period of months (e.g., 3 months or 6 months) or years (e.g., 2 years).

At step 725, the predicted latent vector is decoded at a decoder to generate predicted images. The predicted latent vector generated by the age diffusion module is decoded to generate predicted images at the second time point. Thus, the predicted images generated at step 725 are generated based on the past images encoded at step 715.

At step 730, the current images received at the breast progression predictor at step 710 are compared to the predicted images. In particular, the predicted images are predicted images of healthy breasts that include normal age-related changes from the past images. At step 735, differences between the current images and the predicted images are identified. In some examples, one or more particular portions of the current images can be highlighted as including differences from the predicted images (e.g., the hotspot 610 of FIG. 6A).

At step 740, a likelihood of pathology in the current images is determined based on the differences. In some examples, the likelihood of pathology can be based on a number of differences and/or on a measure of the differences. In some examples, the current images can be encoded as a current image latent vector, and the risk can be based on a distance between the current image latent vector and the healthy breast latent manifold. Thus, when the current image latent vector lies on the healthy breast latent manifold, the risk is zero and the breast is healthy, whereas when current image latent vector lies far from the healthy breast latent manifold, the risk is high. In some examples, the risk can be quantified based on the tangent distance between the healthy breast latent manifold and the current image latent vector.

In some implementations, the current images are encoded to a second latent vector, and, after step 720, the current latent vector is compared to the predicted latent vector. In some examples, a distance between the predicted latent vector and the current latent vector is determined, and the distance is used to identify a risk of a pathological abnormality.

In other implementations, method 700 can be used for data-driven personalized health-tracking of other body parts, and can use a progression predictor for a different body part and/or organ to identify pathological change and/or normal aging of the other body parts. In some examples, the method 700 can be used for data-driven personalized health-tracking of whole body scans. In some implementations, the input images are different types of images, such as x-ray images, CT scan images, MRI images, PET scan images, ultrasound images, radiographic images, elastographic images, photoacoustic images, spectroscopic images, magnetic particle images, or other types of images.

FIG. 7B shows an example of a method 750 for training a data-driven personalized CAD and health-tracking system, according to various examples of the disclosure. The data-driven personalized CAD and health-tracking system can include a breast progression predictor that uses a neural network to identify breast image changes associated with pathologies as well as breast image changes associated with normal aging. In some examples, the breast progression predictor uses a generative model to predict breast image changes associated with normal aging, and identifies pathologies based on differences between changes associated with normal aging and changes detected in input images. As described with respect to FIG. 7B, the breast progression predictor is trained to generate predicted images at a second timepoint, wherein the predicted images are images of healthy breasts and include normal age-related changes as compared to the earlier images of the same breasts. The method 750 may be performed by any of the neural networks described herein. Although the method 750 is described with reference to the flowchart illustrated in FIG. 7B, many other methods for training a data-driven personalized CAD and health-tracking system may alternatively be used. For example, the order of execution of the steps in FIG. 7B may be changed. As another example, some of the steps may be changed, eliminated, or combined.

The system can be trained using a plurality of image pairs for a respective plurality of patients, wherein each image pair includes first and second images, with the first images captured at a first timepoint and the second images captured at a second timepoint. In some examples, the time interval between the first and second images in a respective image pair is one year. In some examples, the time interval is two years, three years, four years, five years, or more than five years. In some examples, the system is trained using many image pairs with different time intervals between the first and second timepoints. In some examples, the system is trained first with image pairs having a one year time interval, then with image pairs having a two year time interval, then with image pairs having a three year time interval, and so on. In various examples, the system is trained with image pairs representing normal aging, such that neither the first images nor the second images include any pathologies such as cancer, cysts, or other pathologies, but the second images differ from the first images due to normal age-related changes in the breasts between the first and second timepoints.

At step 760, an image pair is input to a breast progression predictor, such as the breast progression predictor 300 of FIG. 3. The image pair represents images from a first selected patient. The image pair includes first images and second images, with the second images captured after the first images. In some examples, the second images are captured one year after the first images. In other examples, the second images are captured more than one year after the first images.

At step 765, the first images of the image pair are encoded by the breast progression predictor to generate a first latent vector. At step 770, age diffusion is performed on the first latent vector to generate a predicted second latent vector. In some examples, the first latent vector is input to an age diffusion model. Based on the time interval between the first and second images, the first latent vector undergoes one or more diffusion cycles at the age diffusion model to generate a predicted second latent vector. In one example, there is a one year time interval between the first and second images, and the first latent vector undergoes one cycle of age diffusion to generate the predicted second latent vector. In another example, there is a two year time interval between the first and second images, and the first latent vector undergoes two cycles of age diffusion. In this manner, the age diffusion model generates a predicted second latent vector to correspond with the second images of the input image pair.

At step 775, the predicted second latent vector is decoded to generate predicted images. The predicted images correspond to the type of images input in the image pair. For example, when the first and second images are tomosynthesis images, the predicted images are tomosynthesis images. Here, the input images include first and second images with no pathologies since the input images are being used to train the breast progression predictor to predict normal age-related changes in images of healthy breasts over time. Thus, the second images of the input image pair are the ground-truth images that the model is trained to generate. The goal for the breast progression predictor is to generate predicted images that closely match the second images of the input image pair.

At step 780, the predicted images are compared to the second images, and the training module determines if there are significant differences between the predicted images and the second images. If there are significant differences, the method 750 proceeds to step 785 and the parameters of the model are adjusted based on the differences to update the breast progression predictor to generate predicted images that more closely match the second images (the ground-truth images). Thus, for example, the parameters of the age diffusion module can be updated. In some examples, model weights are adjusted. Once the breast progression predictor is updated at step 785, the method 750 returns to step 770 to perform age diffusion on the first latent vector and generate a predicted second latent vector. Steps 770-785 can be repeated until it is determined that there are not significant differences between the predicted images and the second images.

At step 780, if it is determined that there are not significant differences between the predicted images and the second images, the method 750 returns to step 760, and a next image pair 790 is input to the breast progression predictor for training. In various examples, the breast progression predictor can be trained using a training set including hundreds and/or thousands of pairs of images.

In some examples, the image pairs can include image sequences including first images at a first timepoint, second images at a second timepoint, and third images at a third timepoint. The image sequences can include any number images at any number of timepoints for a selected patient. For instance, image data for a selected patient can include annual images for ten year, twenty years, or more than twenty years. In some examples, the breast progression predictor can be trained to generate predicted images based on more than one previous set of images. In some examples, the age diffusion module can use a first latent vector corresponding to a first timepoint and a second latent vector corresponding to a second timepoint to generate a predicted latent vector corresponding to a third timepoint.

In various implementations, training the breast progression predictor includes generating a healthy breast progression latent space. The breast progression predictor encodes received images into a latent vector and embeds the latent vectors into the healthy breast progression latent space. The breast progression predictor predicts future images by interpolating from a first latent vector along the healthy breast progression latent space to identify a second latent point along the healthy breast progression latent space, and decoding the corresponding second latent vector back to a predicted image. In various implementations, the breast progression latent space can be encoded onto a sequence of points on a smooth manifold, and the entire smooth manifold can be formed during training using the normal aging curves from images of breasts from many different female patients. Thus, the smooth manifold represents a healthy breast latent manifold, and any point on the manifold can be decoded and mapped back to a breast image for a selected patient at a selected age for which the corresponding breast is healthy. Thus, in some examples, a single set of images at a selected timepoint can be encoded to a latent vector, and if the latent vector is not on the healthy breast latent manifold, the images can be flagged by the system as likely containing pathological changes.

In some examples, the personalized CAD and health-tracking system can also be trained to recognize pathological changes. In one example, after the system is trained to identify normal age-related changes (and to generate predicted images including normal age-related changes), the system can be trained to identify pathological changes by identifying images that fall off the healthy breast latent manifold and/or by determining a distance of the encoded latent vector of an input image from the healthy breast latent manifold. In some examples, a threshold can be applied to determine whether an image falls off the healthy breast latent manifold, such that when the distance of the input image from the healthy breast latent manifold exceeds the threshold, the image is identified as potentially having a pathological change.

In some examples, the system can be trained to determine a risk of progression for detected pathological changes. In some examples, the system can be trained to determine a predicted speed of progression of detected pathological changes. For instance, predicted progression of detected pathological changes can be slow. Conversely, predicted progression of detected pathological changes can be fast. In some examples, the speed of progression determination is based on the tangent distance of the image from the healthy breast latent manifold over time. In one example, if a first image at a first timepoint is a first distance from the manifold and a second image at a second timepoint is a second distance from the manifold (where the second distance is greater than the first distance), the speed of progression can be based on the ratio of the difference between the first and second tangent distances divided by the difference between the first and second timepoints:

$$speed of progression = \frac{change in tangent distance}{time gap}$$

In some examples, the risk can be an overall risk for each breast. In some examples, the breast risk can include a separate risk for various regions of each breast.

### Example Neural Network

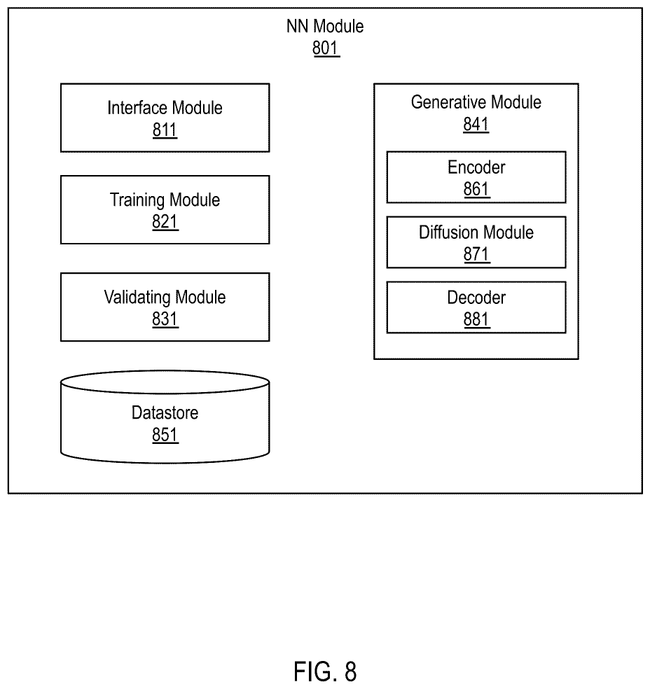

FIG. 8 is a block diagram of a neural network module 801, in accordance with various embodiments. In various examples, the neural network module 801 can be used to implement aspects of the personalized CAD and health-tracking systems described herein. In the embodiments of FIG. 8, the neural network module 801 includes an interface module 811, a training module 821, a validating module 831, a generative model module 841, and a datastore 851. In other embodiments, alternative configurations, different or additional components may be included in the neural network module 801. Further, functionality attributed to a component of the neural network module 801 may be accomplished by a different component included in the neural network module 801 or a different module or system.

The interface module 811 facilitates communications of the neural network module 801 with other modules or systems. For example, the interface module 811 establishes communications between the neural network module 801 with an external database to receive data that can be used to train neural networks or input into neural networks to perform tasks. As another example, the interface module 811 supports the neural network module 801 to distribute neural networks to other systems, e.g., computing devices configured to apply neural networks to perform tasks.

The training module 821 trains neural networks by using a training dataset. The training module 821 forms the training dataset. In some examples, the training module 821 forms the training dataset using sets of input images from patients. The training dataset is used to train the generative model module 841 to generate predicted images at various time intervals that include normal age-related changes, as described, for example, with respect to FIG. 7B. In some embodiments, a part of the training dataset may be used to initially train the neural network, and the rest of the training dataset may be held back as a validation subset used by the validating module 831 to validate performance of a trained neural network. The portion of the training dataset not including the tuning subset and the validation subset may be used to train the neural network. In an embodiment where the training module 821 trains a neural network to recognize pathological hotspots in images, the training dataset includes training images and training labels.

The training labels describe ground truth classifications of pathological hotspots in the training images. In some embodiments, each label in the training dataset corresponds to a pathological hotspot in a training image. In some examples, the labels can include predicted progression risk data.

The training module 821 also determines hyperparameters for training the neural network. Hyperparameters are variables specifying the neural network training process. Hyperparameters are different from parameters inside the neural network (e.g., weights of filters). In some embodiments, hyperparameters include variables determining the architecture of the neural network, such as number of hidden layers, etc. Hyperparameters also include variables which determine how the neural network is trained, such as batch size, number of epochs, etc. A batch size defines the number of training samples to work through before updating the parameters of the neural network. The batch size is the same as or smaller than the number of samples in the training dataset. The training dataset can be divided into one or more batches. The number of epochs defines how many times the entire training dataset is passed forward and backwards through the entire network. The number of epochs defines the number of times that the deep learning algorithm works through the entire training dataset. One epoch means that each training sample in the training dataset has had an opportunity to update the parameters inside the neural network. An epoch may include one or more batches. The number of epochs may be 3, 30, 300, 3000, or even larger.

The training module 821 defines the architecture of the neural network, e.g., based on some of the hyperparameters. In some examples, the architecture of the neural network includes a generative model module 841 having an encoder 861, a diffusion module 871, and a decoder 881. The encoder 861 encodes input images into latent vectors. During training, the diffusion module 871 generates a healthy breast latent manifold based on the way the encoded latent vectors diffuse throughout the latent space. The healthy breast latent manifold can then be used by the diffusion module 871 to predict future latent vectors (for predicted images) at various timepoints based on the manifold. In some examples, the healthy breast latent manifold can be based on a probability distribution of the training dataset. The decoder 881 decodes predicted future latent vectors generated by the diffusion module 871 to produce predicted images.

In some examples, the architecture of the neural network includes an input layer, an output layer, and a plurality of hidden layers. The input layer of a neural network may include tensors (e.g., a multidimensional array) specifying attributes of the input image, such as the height of the input image, the width of the input image, and the depth of the input image (e.g., the number of bits specifying the color of a pixel in the input image). The output layer includes labels of objects, such as pathologies and/or hotspots, in the input layer. The hidden layers are layers between the input layer and output layer. The hidden layers can include one or more convolutional layers and one or more other types of layers, such as pooling layers, fully connected layers, normalization layers, softmax or logistic layers, and so on. The convolutional layers of the neural network abstract the input image to a feature map that is represented by a tensor specifying the feature map height, the feature map width, and the feature map channels (e.g., for a color image, red, green, blue images include 3 channels). A pooling layer is used to reduce the spatial volume of input image after convolution. A pooling layer is used between two convolution layers. A fully connected layer involves weights, biases, and neurons. It connects neurons in one layer to neurons in another layer. It is used to classify images between different categories by training.

In the process of defining the architecture of the neural network, the training module 821 also adds an activation function to a hidden layer or the output layer. An activation function of a layer transforms the weighted sum of the input of the layer to an output of the layer. The activation function may be, for example, a rectified linear unit activation function, a tangent activation function, or other types of activation functions.

After the training module 821 defines the architecture of the neural network, the training module 821 inputs a training dataset into the neural network. The training dataset includes a plurality of training samples. An example of a training sample includes a pathology and/or hotspot in an image and a ground truth label of the pathology and/or hotspot. The training module 821 modifies the parameters inside the neural network (“internal parameters of the neural network”) to minimize the error between labels of the training pathologies and/or hotspots that are generated by the neural network and the ground truth labels of the pathologies and/or hotspots. The internal parameters include weights of filters in the convolutional layers of the neural network. In some embodiments, the training module 821 uses a cost function to minimize the error.

The training module 821 may train the neural network for a predetermined number of epochs. The number of epochs is a hyperparameter that defines the number of times that the deep learning algorithm will work through the entire training dataset. One epoch means that each sample in the training dataset has had an opportunity to update internal parameters of the neural network. After the training module 821 finishes the predetermined number of epochs, the training module 821 may stop updating the parameters in the neural network. The neural network having the updated parameters is referred to as a trained neural network.

The validating module 831 verifies accuracy of trained or compressed neural networks. In some embodiments, the validating module 831 inputs samples in a validation dataset into a trained neural network and uses the outputs of the neural network to determine the model accuracy. In some embodiments, a validation dataset may be formed of some or all the samples in the training dataset. Additionally or alternatively, the validation dataset includes additional samples, other than those in the training sets. In some embodiments, the validating module 831 may determine an accuracy score measuring the precision, recall, or a combination of precision and recall of the neural network. The validating module 831 may use the following metrics to determine the accuracy score: Precision=TP/(TP+FP) and Recall=TP/(TP+FN), where precision may be how many the reference classification model correctly predicted (TP or true positives) out of the total it predicted (TP+FP or false positives), and recall may be how many the reference classification model correctly predicted (TP) out of the total number of pathologies and/or hotspots that did have the property in question (TP+FN or false negatives). The F-score (F-score=2*PR/(P+R)) unifies precision and recall into a single measure.

The validating module 831 may compare the accuracy score with a threshold score. In an example where the validating module 831 determines that the accuracy score of the augmented model is less than the threshold score, the validating module 831 instructs the training module 821 to re-train the neural network. In one embodiment, the training module 821 may iteratively re-train the neural network until the occurrence of a stopping condition, such as the accuracy measurement indication that the neural network may be sufficiently accurate, or a number of training rounds having taken place.

The generative model module 841 performs breast progression prediction as described herein. In the embodiments of FIG. 8, the generative model module 841 includes an image encoder 861, a diffusion module 871, and a decoder 881. In other embodiments, alternative configurations, different or additional components may be included in the generative model module 841. Further, functionality attributed to a component of the generative model module 841 may be accomplished by a different component included in the generative model module 841, the neural network module 801, or a different module or system.

As described herein, encoded latent vector data from the encoder 861 is input to an age diffusion module 871 to generate a predicted latent vector corresponding to a predicted image at a selected timepoint. The output from the diffusion module 871 is input to a decoder 881, configured to decode the predicted latent vector to a predicted image.

The datastore 851 stores data received, generated, used, or otherwise associated with the neural network module 801. For example, the datastore 851 stores the datasets used by the training module 821 and validating module 831. The datastore 851 may also store data generated by the training module 821 and validating module 831, such as the hyperparameters for training neural networks, internal parameters of trained neural networks (e.g., weights, etc.), the healthy breast latent manifold, and so on. In some embodiments the datastore 851 is a component of the neural network module 801. In other embodiments, the datastore 851 may be external to the neural network module 801 and communicate with the neural network module 801 through a network.

### Example Computing Device

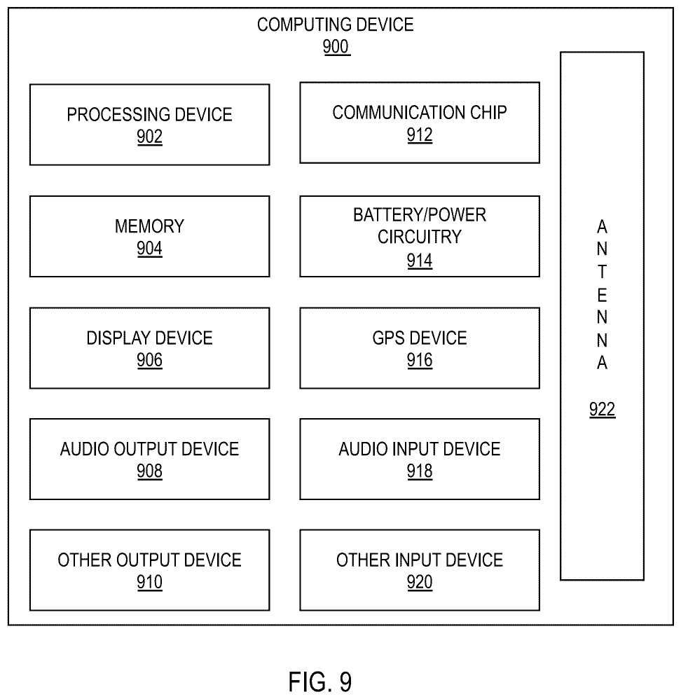

FIG. 9 is a block diagram of an example computing device 900, in accordance with various embodiments. In some embodiments, the computing device 900 can be used as at least part of the health-tracking system 100. The computing device 900 can be used as at least part of a neural network and/or a breast progression predictor. A number of components are illustrated in FIG. 9 as included in the computing device 900, but any one or more of these components may be omitted or duplicated, as suitable for the application. In some embodiments, some or all of the components included in the computing device 900 may be attached to one or more motherboards. In some embodiments, some or all of these components are fabricated onto a single system on a chip (SoC) die. Additionally, in various embodiments, the computing device 900 may not include one or more of the components illustrated in FIG. 9, but the computing device 900 may include interface circuitry for coupling to the one or more components. For example, the computing device 900 may not include a display device 906, but may include display device interface circuitry (e.g., a connector and driver circuitry) to which a display device 906 may be coupled. In another set of examples, the computing device 900 may not include an audio input device 918 or an audio output device 908, but may include audio input or output device interface circuitry (e.g., connectors and supporting circuitry) to which an audio input device 918 or audio output device 908 may be coupled.

The computing device 900 may include a processing device 902 (e.g., one or more processing devices). The processing device 902 processes electronic data from registers and/or memory to transform that electronic data into other electronic data that may be stored in registers and/or memory. The computing device 900 may include a memory 904, which may itself include one or more memory devices such as volatile memory (e.g., DRAM), nonvolatile memory (e.g., read-only memory (ROM)), high bandwidth memory (HBM), flash memory, solid state memory, and/or a hard drive. In some embodiments, the memory 904 may include memory that shares a die with the processing device 902. In some embodiments, the memory 904 includes one or more non-transitory computer-readable media storing instructions executable to perform deep learning operations, e.g., the methods described above in conjunction with FIGS. 7A-7B. The instructions stored in the one or more non-transitory computer-readable media may be executed by the processing device 902.

In some embodiments, the computing device 900 may include a communication chip 912 (e.g., one or more communication chips). For example, the communication chip 912 may be configured for managing wireless communications for the transfer of data to and from the computing device 900. The term “wireless” and its derivatives may be used to describe circuits, devices, systems, methods, techniques, communications channels, etc., that may communicate data through the use of modulated electromagnetic radiation through a nonsolid medium. The term does not imply that the associated devices do not contain any wires, although in some embodiments they might not.

The communication chip 912 may implement any of a number of wireless standards or protocols, including but not limited to Institute for Electrical and Electronic Engineers (IEEE) standards including Wi-Fi (IEEE 802.10 family), IEEE 802.16 standards (e.g., IEEE 802.16-2005 Amendment), Long-Term Evolution (LTE) project along with any amendments, updates, and/or revisions (e.g., advanced LTE project, ultramobile broadband (UMB) project (also referred to as “3GPP2”), etc.). IEEE 802.16 compatible Broadband Wireless Access (BWA) networks are generally referred to as WiMAX networks, an acronym that stands for worldwide interoperability for microwave access, which is a certification mark for products that pass conformity and interoperability tests for the IEEE 802.16 standards. The communication chip 912 may operate in accordance with a Global System for Mobile Communication (GSM), General Packet Radio Service (GPRS), Universal Mobile Telecommunications System (UMTS), High Speed Packet Access (HSPA), Evolved HSPA (E-HSPA), or LTE network. The communication chip 912 may operate in accordance with Enhanced Data for GSM Evolution (EDGE), GSM EDGE Radio Access Network (GERAN), Universal Terrestrial Radio Access Network (UTRAN), or Evolved UTRAN (E-UTRAN). The communication chip 912 may operate in accordance with Code-division Multiple Access (CDMA), Time Division Multiple Access (TDMA), Digital Enhanced Cordless Telecommunications (DECT), Evolution-Data Optimized (EV-DO), and derivatives thereof, as well as any other wireless protocols that are designated as 3G, 4G, 5G, and beyond. The communication chip 912 may operate in accordance with other wireless protocols in other embodiments. The computing device 900 may include an antenna 922 to facilitate wireless communications and/or to receive other wireless communications (such as AM or FM radio transmissions).

In some embodiments, the communication chip 912 may manage wired communications, such as electrical, optical, or any other suitable communication protocols (e.g., the Ethernet). As noted above, the communication chip 912 may include multiple communication chips. For instance, a first communication chip 912 may be dedicated to shorter-range wireless communications such as Wi-Fi or Bluetooth, and a second communication chip 912 may be dedicated to longer-range wireless communications such as global positioning system (GPS), EDGE, GPRS, CDMA, WiMAX, LTE, EV-DO, or others. In some embodiments, a first communication chip 912 may be dedicated to wireless communications, and a second communication chip 912 may be dedicated to wired communications.

The computing device 900 may include battery/power circuitry 914. The battery/power circuitry 914 may include one or more energy storage devices (e.g., batteries or capacitors) and/or circuitry for coupling components of the computing device 900 to an energy source separate from the computing device 900 (e.g., AC line power).

The computing device 900 may include a display device 906 (or corresponding interface circuitry, as discussed above). The display device 906 may include any visual indicators, such as a heads-up display, a computer monitor, a projector, a touchscreen display, a liquid crystal display (LCD), a light-emitting diode display, or a flat panel display, for example.

The computing device 900 may include an audio output device 908 (or corresponding interface circuitry, as discussed above). The audio output device 908 may include any device that generates an audible indicator, such as speakers, headsets, or earbuds, for example.

The computing device 900 may include an audio input device 918 (or corresponding interface circuitry, as discussed above). The audio input device 918 may include any device that generates a signal representative of a sound, such as microphones, microphone arrays, or digital instruments (e.g., instruments having a musical instrument digital interface (MIDI) output).

The computing device 900 may include a GPS device 916 (or corresponding interface circuitry, as discussed above). The GPS device 916 may be in communication with a satellite-based system and may receive a location of the computing device 900, as known in the art.

The computing device 900 may include another output device 910 (or corresponding interface circuitry, as discussed above). Examples of the other output device 910 may include an audio codec, a video codec, a printer, a wired or wireless transmitter for providing information to other devices, or an additional storage device.

The computing device 900 may include another input device 920 (or corresponding interface circuitry, as discussed above). Examples of the other input device 920 may include an accelerometer, a gyroscope, a compass, an image capture device, a keyboard, a cursor control device such as a mouse, a stylus, a touchpad, a bar code reader, a Quick Response (QR) code reader, any sensor, or a radio frequency identification (RFID) reader.

The computing device 900 may have any desired form factor, such as a handheld or mobile computer system (e.g., a cell phone, a smart phone, a mobile internet device, a music player, a tablet computer, a laptop computer, a netbook computer, an ultrabook computer, a personal digital assistant (PDA), an ultramobile personal computer, etc.), a desktop computer system, a server or other networked computing component, a printer, a scanner, a monitor, a set-top box, an entertainment control unit, a vehicle control unit, a digital camera, a digital video recorder, or a wearable computer system. In some embodiments, the computing device 900 may be any other electronic device that processes data.

### SELECTED EXAMPLES

The following paragraphs provide various examples of the embodiments disclosed herein.

Example 1 provides a method for differentiating pathological change from normal aging in breast images, including receiving input breast images at a breast progression predictor, where the input breast images include first images captured at a first timepoint and second images captured at a second timepoint; encoding the first images to a first latent vector; generating a predicted latent vector at an age diffusion module, based on a healthy breast latent space, the first latent vector, and a time interval between the first timepoint and the second timepoint; decoding the predicted latent vector to generate predicted images at the second timepoint; identifying differences between the second images and the predicted images; and determining, based on the differences, a likelihood of pathology in the second images.

Example 2 provides the method, system, apparatus, or neural network of any of the previous and/or following examples, wherein generating the predicted latent vector at the age diffusion module includes determining a number of diffusion cycles based on the time interval and performing the number of diffusion cycles on the first latent vector.

Example 3 provides the method, system, apparatus, or neural network of any of the previous and/or following examples, further including encoding the second images to a second latent vector.

Example 4 provides the method, system, apparatus, or neural network of any of the previous and/or following examples, further including determining a distance between the second latent vector and the predicted latent vector.

Example 5 provides the method, system, apparatus, or neural network of any of the previous and/or following examples, where determining the likelihood of pathology includes determining the likelihood based on the distance, where a greater distance value indicates a greater risk value.

Example 6 provides the method, system, apparatus, or neural network of any of the previous and/or following examples, where the likelihood of pathology in the second images includes a risk of cancer.

Example 7 provides the method, system, apparatus, or neural network of any of the previous and/or following examples, further including displaying the predicted images and the second images in a user interface.

Example 8 provides the method, system, apparatus, or neural network of any of the previous and/or following examples, further including identifying an area of pathological change in the second images and highlighting the area in the second images in the user interface.

Example 9 provides the method, system, apparatus, or neural network of any of the previous and/or following examples, further including receiving multimodality patient data at the breast progression predictor, and embedding the multimodality patient data in a third latent vector.

Example 10 provides the method, system, apparatus, or neural network of any of the previous and/or following examples, where generating the predicted latent vector further includes generating the predicted latent vector based on the third latent vector.

Example 11 provides a system for differentiating pathological change from normal aging in breast images, including a breast progression predictor configured to receive input breast images, including first images captured at a first timepoint and second images captured at a second timepoint, the breast progression predictor including: an encoder configured to encode the first images to a first latent vector, a healthy breast latent manifold representing normal breast image latent space at various ages, an age diffusion module configured to generate a predicted latent vector based on the healthy breast latent manifold, the first latent vector, and a time interval between the first timepoint and the second timepoint, and a decoder configured to decode the predicted latent vector to generate predicted images at the second timepoint; where the breast progression predictor is further configured to: identify differences between the second images and the predicted images, and determine, based on the differences, a likelihood of pathology in the second images.

Example 12 provides the method, system, apparatus, or neural network of any of the previous and/or following examples, the age diffusion module is further configured to: determine a number of diffusion cycles based on the time interval, and perform the number of diffusion cycles on the first latent vector to generate the predicted latent vector.

Example 13 provides the method, system, apparatus, or neural network of any of the previous and/or following examples, where the encoder is further configured to encode the second images to a second latent vector.

Example 14 provides the method, system, apparatus, or neural network of any of the previous and/or following examples, where the breast progression predictor is further configured to: determine a distance between the second latent vector and the predicted latent vector, and determine the likelihood of pathology based on the distance, where a greater distance value indicates a greater risk value.

Example 15 provides the method, system, apparatus, or neural network of any of the previous and/or following examples, where the breast progression predictor is further configured to determine a predicted speed of progression of the pathology.

Example 16 provides the method, system, apparatus, or neural network of any of the previous and/or following examples, where the breast progression predictor is further configured to identify an area of pathological change in the second images, and further including a user interface configured to display the predicted images and the second images, and to highlight the area of pathological change in the second images.

Example 17 provides the method, system, apparatus, or neural network of any of the previous and/or following examples, where the encoder is a first encoder and further including a second encoder, where the breast progression predictor is further configured to receive multimodality patient data, and the second encoder is configured to encode the multimodality patient data in a third latent vector.

Example 18 provides the method, system, apparatus, or neural network of any of the previous and/or following examples, where the breast progression predictor is further configured to generate the predicted latent vector based on the third latent vector.

Example 19 provides a method for differentiating pathological change from normal aging in breast images, comprising: receiving input breast images at a breast progression predictor, wherein the input breast images include first images captured at a first timepoint and second images captured at a second timepoint; encoding the first images to a first latent vector; encoding the second images to a second latent vector; generating a predicted latent vector at an age diffusion module, based on a healthy breast latent manifold, the first latent vector, and a time interval between the first timepoint and the second timepoint; determining a distance between the second latent vector and the predicted latent vector; and determining, based on the distance, a likelihood of pathology in the second images.

Example 20 provides the method, system, apparatus, or neural network of any of the previous and/or following examples, further comprising determining, based on the distance, a speed of progression of an identified pathological abnormality.

Example 21 provides a neural network for differentiating pathological change from normal aging in breast images, including a receiver configured to receive input breast images, including first images captured at a first timepoint and second images captured at a second timepoint; an encoder configured to encode the first images to a first latent vector; a healthy breast latent manifold representing normal breast image latent space at various ages; an age diffusion module configured to generate a predicted latent vector based on the healthy breast latent manifold, the first latent vector, and a time interval between the first timepoint and the second timepoint; a decoder configured to decode the predicted latent vector to generate predicted images at the second timepoint; a risk determination module configured to identify differences between the second images and the predicted images, and determine, based on the differences, a likelihood of pathology in the second images.

Example 22 provides the neural network of example 19, where the age diffusion module is further configured to: determine a number of diffusion cycles based on the time interval, and perform the number of diffusion cycles on the first latent vector to generate the predicted latent vector.

Example 23 provides the method, system, apparatus, or neural network of any of the previous and/or following examples, wherein conditional image generation is used to combine the first latent vector with the third latent vector.

Example 24 provides the method, system, apparatus, or neural network of any of the previous and/or following examples, wherein the breast progression predictor is a neural network.

Example 25 provides the method, system, apparatus, or neural network of any of the previous and/or following examples, wherein the breast progression predictor is a generative model.

Example 26 provides one or more non-transitory computer-readable media storing instructions executable to perform operations, the operations comprising: receiving input breast images at a breast progression predictor, where the input breast images include first images captured at a first timepoint and second images captured at a second timepoint; encoding the first images to a first latent vector; generating a predicted latent vector at an age diffusion module, based on a healthy breast latent space, the first latent vector, and a time interval between the first timepoint and the second timepoint; decoding the predicted latent vector to generate predicted images at the second timepoint; identifying differences between the second images and the predicted images; and determining, based on the differences, a likelihood of pathology in the second images.

Example 27 provides an apparatus, comprising: a computer processor for executing computer program instructions; and a non-transitory computer-readable memory storing computer program instructions executable by the computer processor to perform operations comprising receiving input breast images at a breast progression predictor, where the input breast images include first images captured at a first timepoint and second images captured at a second timepoint; encoding the first images to a first latent vector; generating a predicted latent vector at an age diffusion module, based on a healthy breast latent space, the first latent vector, and a time interval between the first timepoint and the second timepoint; decoding the predicted latent vector to generate predicted images at the second timepoint; identifying differences between the second images and the predicted images; and determining, based on the differences, a likelihood of pathology in the second images.

Example 28 provides a method for training a neural network to differentiate pathological change from normal aging in breast images, comprising: inputting the image pair to a breast progression predictor, wherein the image pair includes first images and second images, and wherein the second images are captured after the first images, encoding the first images of the image pair to a first latent vector, performing age diffusion on the first latent vector to generate a predicted second latent vector, decoding the predicted second latent vector to generate predicted images, identifying significant differences between the predicted images and the second images, and updating the breast progression predictor based on the differences.

Example 29 provides the method, system, apparatus, or neural network of any of the previous and/or following examples, further comprising constructing a breast progression latent space for healthy breast images based on encoded latent vector representations of healthy breast images.

Example 30 provides the method, system, apparatus, or neural network of any of the previous and/or following examples, wherein determining a likelihood of pathology includes determining a speed of pathological progression.

Example 31 provides a method for differentiating pathological change from normal aging in breast images, including receiving input breast images at a breast progression predictor, where the input breast images include first images captured at a first timepoint and second images captured at a second timepoint; encoding the first images to a first latent vector and second images to a second latent vector; generating a predicted future latent vector at an age diffusion module, based on a breast latent space, the first and second latent vectors, and a time interval between the first timepoint and the second timepoint; determining a distance between a healthy breast latent space and the predicted future latent vector; and determining, based on the distance, a likelihood of future pathological abnormalities.

Example 32 provides a neural network for differentiating pathological change from normal aging in breast images, comprising a receiver configured to receive input breast images, including first images captured at a first timepoint and second images captured at a second timepoint, an encoder configured to encode the first images to a first latent vector, a healthy breast latent manifold representing normal breast image latent space at various ages, an age diffusion module configured to generate a predicted latent vector based on the healthy breast latent manifold, the first latent vector, and a time interval between the first timepoint and the second timepoint, a decoder configured to decode the predicted latent vector to generate predicted images at the second timepoint, and a risk determination module configured to identify differences between the second images and the predicted images, and determine, based on the differences, a likelihood of pathology in the second images.

Example 33 provides a neural network according to example 32, wherein the age diffusion module is further configured to determine a number of diffusion cycles based on the time interval, and perform the number of diffusion cycles on the first latent vector to generate the predicted latent vector.

The above description of illustrated implementations of the disclosure, including what is described in the Abstract, is not intended to be exhaustive or to limit the disclosure to the precise forms disclosed. While specific implementations of, and examples for, the disclosure are described herein for illustrative purposes, various equivalent modifications are possible within the scope of the disclosure, as those skilled in the relevant art will recognize. These modifications may be made to the disclosure in light of the above detailed description.

1. A method for differentiating pathological change from normal aging in breast images, comprising:
receiving input breast images at a breast progression predictor, wherein the input breast images include first images captured at a first timepoint and second images captured at a second timepoint;
encoding the first images to a first latent vector;
generating a predicted latent vector at an age diffusion module, based on a healthy breast latent space, the first latent vector, and a time interval between the first timepoint and the second timepoint;
decoding the predicted latent vector to generate predicted images at the second timepoint;
identifying a difference between the second images and the predicted images; and
determining, based on the difference, a likelihood of pathology in the second images.

2. The method of claim 1, wherein generating the predicted latent vector at the age diffusion module comprises determining a number of diffusion cycles based on the time interval and performing the number of diffusion cycles on the first latent vector.

3. The method of claim 1, further comprising encoding the second images to a second latent vector.

4. The method of claim 3, further comprising determining a distance between the second latent vector and the predicted latent vector.

5. The method of claim 4, wherein determining the likelihood of pathology includes determining the likelihood based on the distance, wherein a greater distance value indicates a greater risk value.

6. The method of claim 1, wherein the likelihood of pathology in the second images includes a risk of cancer.

7. The method of claim 1, further comprising displaying the predicted images and the second images in a user interface.

8. The method of claim 7, further comprising identifying an area of pathological change in the second images and highlighting the area in the second images in the user interface.

9. The method of claim 1, further comprising receiving multimodality patient data at the breast progression predictor, and embedding the multimodality patient data in a third latent vector.

10. The method of claim 9, wherein generating the predicted latent vector further comprises generating the predicted latent vector based on the third latent vector.

11. A system for differentiating pathological change from normal aging in breast images, comprising:
a breast progression predictor configured to receive input breast images, including first images captured at a first timepoint and second images captured at a second timepoint, the breast progression predictor including:
an encoder configured to encode the first images to a first latent vector,
a healthy breast latent manifold representing normal breast image latent space at various ages,
an age diffusion module configured to generate a predicted latent vector based on the healthy breast latent manifold, the first latent vector, and a time interval between the first timepoint and the second timepoint, and
a decoder configured to decode the predicted latent vector to generate predicted images at the second timepoint;
wherein the breast progression predictor is further configured to:
identify a difference between the second images and the predicted images, and
determine, based on the difference, a likelihood of pathology in the second images.

12. The system of claim 11, wherein the age diffusion module is further configured to:
determine a number of diffusion cycles based on the time interval, and
perform the number of diffusion cycles on the first latent vector to generate the predicted latent vector.

13. The system of claim 11, wherein the encoder is further configured to encode the second images to a second latent vector.

14. The system of claim 13, wherein the breast progression predictor is further configured to:
determine a distance between the second latent vector and the predicted latent vector, and
determine the likelihood of pathology based on the distance, wherein a greater distance value indicates a greater risk value.

15. The system of claim 14, wherein the breast progression predictor is further configured to determine a predicted speed of progression of the pathology.

16. The system of claim 11, wherein the breast progression predictor is further configured to identify an area of pathological change in the second images, and further comprising a user interface configured to display the predicted images and the second images, and to highlight the area of pathological change in the second images.

17. The system of claim 11, wherein the encoder is a first encoder and further comprising a second encoder, wherein the breast progression predictor is further configured to receive multimodality patient data, and the second encoder is configured to encode the multimodality patient data in a third latent vector.

18. The system of claim 17, wherein the breast progression predictor is further configured to generate the predicted latent vector based on the third latent vector.

19. The system of claim 11, wherein the breast progression predictor is further configured to receive multimodality patient data, and wherein the encoder is further configured to embed the multimodality patient data in a third latent vector.

20. An apparatus, comprising:
a computer processor for executing computer program instructions; and
a non-transitory computer-readable memory storing computer program instructions executable by the computer processor to perform operations comprising:
receiving input breast images at a breast progression predictor, where the input breast images include first images captured at a first timepoint and second images captured at a second timepoint;
encoding the first images to a first latent vector;
generating a predicted latent vector at an age diffusion module, based on a healthy breast latent space, the first latent vector, and a time interval between the first timepoint and the second timepoint;
decoding the predicted latent vector to generate predicted images at the second timepoint;
identifying differences between the second images and the predicted images; and
determining, based on the differences, a likelihood of pathology in the second images.
